(19) United States
(12) Patent Application Publication
(10) Pub. No.: US 2008/0025960 A1
(43) Pub. Date: Jan. 31, 2008

(54) Detergents with stabilized enzyme systems

(76) Inventors: Manoj Kumar, Fremont, CA (US); Ayrookaran J. Poulose, Belmont, CA (US)

Correspondence Address: KAMRIN T. MacKNIGHT;Danisco US Inc., Genencor Division, 925 Page Mill Road, Palo Alto, CA, 94304-1013, US

(21) Appl. No.: 11/825,229

(22) Filed: Jul. 5, 2007

Related U.S. Application Data
(60) Provisional application No. 60/818,824, filed on Jul. 6, 2006.

(51) Int. Cl. C11D 7/42; A61K 38/43; A61P 43/00

(52) U.S. Cl. 424/94.3; 510/392; 510/393; 8/401

(57) ABSTRACT

The present invention provides methods and compositions for the stabilization of oxidase enzymes during storage. In some preferred embodiments, the oxidase is a component of liquid detergent compositions. In some particularly preferred embodiments, the oxidase is stabilized by the addition of a reversible inhibitor of the oxidase to a liquid detergent. In some particularly preferred embodiments, the oxidase is stabilized with bisulfite. In further preferred embodiments, the use of a reversible inhibitor also prevents premature generation of peroxide during storage of liquid detergent. In additional embodiments, liquid detergent formulations comprised of oxidase enzyme, its substrate, and its reversible inhibitor produce active oxygen species (peroxide) upon dilution of the liquid detergent in laundry wash liquor.

The present application claims priority to pending U.S. Provisional Patent Application Ser. No. 60/818,824, filed Jul. 6, 2006.

### FIELD OF THE INVENTION

The present invention provides methods and compositions for the stabilization of oxidase enzymes during storage. In some preferred embodiments, the oxidase is a component of liquid compositions that further comprise at least one oxidase substrate. In some preferred embodiments, the oxidase is a component of liquid detergent compositions. In some particularly preferred embodiments, the oxidase is stabilized by the addition of a reversible inhibitor of the oxidase to a liquid detergent. In some particularly preferred embodiments, the oxidase is stabilized with bisulfite. In further preferred embodiments, the use of a reversible inhibitor also prevents premature generation of peroxide during storage of liquid detergent. In additional embodiments, liquid detergent formulations comprised of oxidase enzyme, its substrate, and its reversible inhibitor produce active oxygen species (peroxide) upon dilution of the liquid detergent in laundry wash liquor.

### BACKGROUND OF THE INVENTION

Detergents for laundry and dish washing consist of complex mixtures of a wide variety of ingredients, which typically include a number of components such as ionic and non-ionic surfactants, solvents, builders, perfumes, enzymes, and bleaching components. In such complex mixtures, storage stability problems, particularly of enzymes, are well known. In some cases, stability problems are related to the physical stability of the detergent, while in other cases, it relates to the functional stability of the individual ingredients in the detergent. Enzymes such as oxidases are in particular susceptible to storage stability issues in liquid detergent formulation. This prevents their widespread use in fabric and household cleaning compositions that involve bleaching action. Maintaining the oxidase enzymatic activity in detergents during storage has been a challenge, especially in detergents that also contain oxidase substrate components. The presence of both oxidase and oxidase substrate results in the in situ generation of hydrogen peroxide. This results in decreased enzyme stability due to oxidation of the enzymes both in liquid and dry formulations. It is contemplated that peroxide damage to enzymes occurs by various mechanisms (e.g., oxidation of key amino acid residues in the enzyme by interacting with the enzymes' cofactors etc.). However, it is not intended that the present invention be limited to any particular mechanism. Nonetheless, peroxide damage to enzymes often results in a gradual loss of activity. In dry detergent formulations enzymes can be stabilized by (e.g. encapsulation of the enzymes as described in WO 96/02623, incorporated herein by reference in its entirety).

Various laundry bleaches and activators are known in the art (See e.g., Grime and Clauss, Chem. Indust., 20:647-649, 652-653 [1990]; Sheane and Wilkinson, Tinctoria 101:36-41[2004]; and Broze, Handbook of Detergents, Warwick International, [1999]). The most commonly used bleaching agents include sodium perborate, sodium percarbonate, sodium persulfate, sodium perphosphate, urea peroxide, sodium persilicate, their ammonium, potassium and lithium analogs, calcium peroxide, zinc peroxide, sodium peroxide, carbamide peroxide, and others such as sodium hypochlorite and chlorine oxide are commonly used in detergents, toothpastes, and other products. This peroxide oxidizing power at a low temperature can be elevated by adding a “bleaching activator.” Varieties of bleaching activators are known in the art and include acyl compounds such as tetraacetylethylenediamine (TAED), ester compounds such as nonanoyloxybenzenesulfonate (NOBS) and isononanoyloxybenzenesulfonate (ISONOBS), transition metal complexes, and other compounds.

This bleaching system generates peracids (e.g., peracetic acid), hydrogen peroxide, and/or other related species upon addition of water during the wash cycle. The peracids and the other active oxygen species present in the system then act to bleach or lighten certain stains on the fabric or dishware. However, bleach activators cannot be added with percarbonate in liquid detergents, since they will react and form peracids and/or other activated oxidizing agents. Thus, there is a need for an H₂O₂ generating system that is inactive during storage, but generates hydrogen peroxide during the wash cycle.

Bleaching agents are typically not included in liquid detergents due to poor storage stability of the bleaching agents in detergents that contain significant amounts of water (e.g., more than 1% water). The presence of bleaching agents also greatly negatively impacts the storage stability of oxidatively sensitive enzymes and other compounds included in detergents. Thus, there is a need for liquid detergents that provide in situ generation of bleaching agents upon dilution of the detergent in wash liquor.

Several oxidases have been described (See e.g., Beck et al., Bleach activators. Carbohydrates as Organic Raw Materials III, developed from a Workshop, Wageningen, Nov. 28-29, 1994, pages 295-306 [1996]; Nakayama and Amachi, J. Mol. Catalysis B: Enzymatic 6:185-198 [1999]; WO 06/008497; WO 05/124012; U.S. Pat. No. 6,399,329; WO 01/007555; and WO 03/36094. However the major limitation of these systems is that when oxidases along with their substrates are stored in the liquid detergent, they produce hydrogen peroxide, which by itself can damage enzymes and also can react with the bleach activators present in the system. Thus, such oxidase substrate systems are unstable. Indeed, there remains a need in the art for means to provide reversibly inhibited oxidases in the presence of substrates during storage that will produce in situ bleaching agents when diluted into the wash liquor. In addition, there is a need for the production of bleaching agents (e.g., active oxygen species, peroxide, and peracids) upon dilution of the detergent in the laundry wash liquor to bleach and/or lighten stains.

As with liquids, the presence of bleaching agents in detergent powders often has strong negative effects on the stability of enzymes present in the detergent. Consequently, great care is taken to separate the enzyme molecules and the bleaching agents in the detergent powder. This is usually accomplished by separately formulating the enzymes and the bleaching agents. For example, in some cases, the enzymes are formulated in granulates prepared in such a way as to reduce the penetration of active oxygen species into enzyme-containing granules during storage Such powder detergent systems can also benefit from a reversibly inhibited oxidase substrate enzyme system.

### SUMMARY OF THE INVENTION

The present invention provides methods and compositions for the stabilization of oxidase enzymes during storage. In some preferred embodiments, the oxidase is a component of liquid detergent compositions. In some particularly preferred embodiments, the oxidase is stabilized by the addition of a reversible inhibitor of the oxidase to a liquid detergent. In some particularly preferred embodiments, the oxidase is stabilized with bisulfite. In further preferred embodiments, the use of a reversible inhibitor also prevents premature generation of peroxide during storage of liquid detergent. In additional embodiments, liquid detergent formulations comprised of oxidase enzyme, its substrate, and its reversible inhibitor produce active oxygen species (peroxide) upon dilution of the liquid detergent in laundry wash liquor. In additional embodiments, the present invention provides powder detergent formulations comprised of at least one oxidase enzyme, at least one oxidase substrate, and at least one reversible inhibitor. In some particularly preferred embodiments, these powder detergent formulations produce active oxygen species (peroxide) upon dilution of the powder detergent in laundry wash liquor.

The present invention provides stabilized oxidase compositions comprising at least one oxidase and at least one stabilizer. In some embodiments, the oxidase is selected from glucose oxidase, sorbitol oxidase, choline oxidase, hexose oxidase, and alcohol oxidase. In some alternative embodiments, the compositions further comprise at least one substrate for the at least one oxidase. In some preferred embodiments, the substrate is selected from glucose, lactate, sorbitol, choline, glycerol, ethylene glycol, propylene glycol, and ethanol. In some alternative embodiments, the at least one stabilizer comprises at least one oxidase inhibitor. In some preferred embodiments, the stabilizer comprises at least one sulfite. In some particularly preferred embodiments, the at least one sulfite is selected from sodium hydrogen sulfite, sodium metabisulfite, and/or sodium bisulfite. In some alternative preferred embodiments, the stabilizer is selected from thiosulfate and 2-amino-2 methyl-1-propanol. In some particularly preferred embodiments, the composition is a cleaning, bleaching and/or disinfecting composition. In some alternative preferred embodiments, the detergent is a laundry detergent or a dish detergent. In some further embodiments, the detergent is selected from powder, liquid and gel detergents. In some yet additional embodiments, the composition is a detergent additive or a pretreatment product. In some still further embodiments, the composition further comprises a bleach activator or a bleach precursor. In some embodiments, the bleach activator is selected from peracid precursors, metal complexes, peroxidases, and an acyl transferase-substrate system. In some particularly preferred embodiments, the compositions further comprise at least one enzyme selected from proteases, amylases, pectinases, pectate lyases, lipases, mannanases, cellulases, esterases, cutinases, oxidoreductases, hemicellulases, and carbohydrases. In some additional embodiments, the compositions further comprise at least one adjunct ingredient selected from surfactants, builders, whitening agents, antimicrobial agents, polymers, solvents, salts, buffering agents, chelating agents, dye transfer inhibiting agents, deposition aids, dispersants, enzymes, enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, pigments and mixtures thereof.

The present invention also provides methods for producing bleach species in a wash liquor comprising the step of adding at least one composition of the present invention to the wash liquor. In yet additional embodiments, the bleaching species is peroxide or a bleaching system that can be activated by peroxide.

### BRIEF DESCRIPTION OF THE DRAWINGS

FIG. 1 provides a graph showing the effect of bisulfite on the stability and bleaching performance of glucose oxidase.

FIG. 2 provides a graph showing the effect of bisulfite on the stability and bleaching performance of glucose oxidase on blueberry-stained disks.

FIG. 3 provides a graph showing the effect of bisulfite on the stability and bleaching performance of glucose oxidase on multiple stained swatches tested in a tergotometer.

### DESCRIPTION OF THE INVENTION

The present invention provides methods and compositions for the stabilization of oxidase enzymes during storage. In some preferred embodiments, the oxidase is a component of liquid detergent compositions. In some particularly preferred embodiments, the oxidase is stabilized by the addition of a reversible inhibitor of the oxidase to a liquid detergent. In further preferred embodiments, the use of a reversible inhibitor also prevents premature generation of peroxide during storage of liquid detergent. In additional embodiments, liquid detergent formulations comprised of oxidase enzyme, its substrate, and its reversible inhibitor produce active oxygen species (peroxide) upon dilution of the liquid detergent in laundry wash liquor. In some particularly preferred embodiments, the oxidase is stabilized with bisulfite. In additional embodiments, the present invention provides liquid detergent formulations comprised of at least one oxidase at least one oxidase substrate, and at least one reversible inhibitor. In particularly preferred embodiments, these liquid detergent formulations produce active oxygen species (e.g., peroxide) upon dilution of the liquid detergent in laundry wash liquor.

Definitions

Unless otherwise indicated, the practice of the present invention involves conventional techniques commonly used in molecular biology, microbiology, protein purification, protein engineering, protein and DNA sequencing, recombinant DNA fields, and industrial enzyme use and development, all of which are within the skill of the art. All patents, patent applications, articles and publications mentioned herein, both supra and infra, are hereby expressly incorporated herein by reference.

Furthermore, the headings provided herein are not limitations of the various aspects or embodiments of the invention, which can be had by reference to the specification as a whole. Accordingly, the terms defined immediately below are more fully defined by reference to the specification as a whole. Nonetheless, in order to facilitate understanding of the invention, definitions for a number of terms are provided below.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “oxidase” refers to enzymes that catalyze an oxidation/reduction reaction involving molecular oxygen (O₂) as the electron acceptor. In these reactions, oxygen is reduced to water (H₂O) or hydrogen peroxide (H₂O₂). The oxidases are a subclass of the oxidoreductases.

As used herein, the term “glucose oxidase” (“Gox”) refers to the oxidase enzyme (EC 1.1.3.4) which catalyzes the oxidation of beta-D-glucose into D-glucono-1,5-lactone, which then hydrolyzes to gluconic acid with concomitant reduction of molecular oxygen to hydrogen peroxide.

As used herein, the term “alcohol oxidase” (“Aox”) refers to the oxidase enzyme (EC 1.1.3.13) that converts an alcohol to an aldehyde with concomitant reduction of molecular oxygen to hydrogen peroxide.

As used herein, the term “choline oxidase” (“Cox”) refers to an oxidase enzyme (EC 1.1.3. 17) that catalyzes the four-electron oxidation of choline to glycine betaine, with betaine aldehyde as an intermediate with concomitant reduction of two molecules of molecular oxygen to two molecules of hydrogen peroxide.

As used herein, the term “hexose oxidase” (“Hox”) refers to an oxidase enzyme (EC 1.1.3.5) the oxidation of mono- and disaccharides to their corresponding lactones, with concomitant reduction of molecular oxygen to hydrogen peroxide. Hexose oxidase is able to oxidize a variety of substrates including D-glucose, D-galactose, maltose, cellobiose, and lactose, etc. It is not intended that the present invention be limited to any particular hexose.

As used herein, “glycerol oxidase” refers to an oxidase enzyme (EC 1.1.3.) that catalyzes the oxidation of glycerol to glyceraldehyde, with concomitant reduction of molecular oxygen to hydrogen peroxide.

As used herein, “sorbitol oxidase” refers to a polyol oxidase enzyme (EC 1.1.3.) that catalyzes the oxidation of a substrate (e.g., D-sorbitol) to D-glucose, with concomitant reduction of molecular oxygen to hydrogen peroxide. The substrates for sorbitol oxidase also include various polyols (e.g., xylitol, arabitol, mannitol, ribitol, glycerol, propanediol, and propylene glycol). As used herein, “polyol” refers to chemical compounds that contain multiple hydroxyl groups.

Additional oxidases find use in the present invention, including but not limited to cholesterol oxidase, pyranose oxidase, carboxyalcohol oxidase, L-amino acid oxidase, glycine oxidase, pyruvate oxidase, glutamate oxidase, sarcosine oxidase, lysine oxidase, lactate oxidase, vanillyl oxidase, glycolate oxidase, galactose oxidase, uricase, oxalate oxidase, xanthine oxidase.

As used herein, “inhibitors” refers to chemical compounds that can reduce or stop the catalytic activity of an enzyme. In particularly preferred embodiments, the inhibitors reduce or stop the catalytic activity of at least one oxidase. Examples of oxidase inhibitors include acetate, silver salts, halide ions, sec- and tert-alcohols, isocyanate, isothiocyante, glucose analogs, bisulfite, sulfite, thiosulfate, metabisulfite, zinc salts, diethyl dicarbamate, methyl methane sulfonate, acrylonitrile, 2-amino,2-methyl 1-propanol.

As used herein, “reversible enzyme inhibitor” refers to molecules that bind to an enzyme and decrease its rate of reaction. In some embodiments, reversible enzyme inhibitors are affected by varying the concentration of the enzyme's substrate in relation to the inhibitor. In some embodiments, reversible enzyme inhibitors bind to the enzyme using weak bonds that are similar to those used to bind to substrate. Thus, the reversible inhibitor does not permanently disable the enzyme, as removal of the inhibitor allows the enzyme to bind to and turnover its substrate. In some embodiments, reversible enzyme inhibitors are competitive inhibitors that interact non-covalently with the enzyme, and/or compete with the substrate for the enzyme's active site, and/or have structures that are similar to the substrate, products and/or transition state. In additional embodiments, the reversible inhibitor is a non-competitive enzyme inhibitor that binds at a site present on the enzyme other than the active site, and/or causes conformational changes in the enzyme that decrease, and/or stop catalytic activity. It is not intended that the term be limited to any particular mechanism or type of reversible enzyme inhibitor. It is only necessary that the effects of the enzyme inhibitor be reversible, such that the enzyme will function in the absence of the inhibitor and/or the effects of the inhibitor.

As used herein, the term “compatible,” means that the cleaning composition materials do not reduce the enzymatic activity of the oxidase enzyme(s) provided herein to such an extent that the oxidases(s) is/are not effective as desired during normal use situations. Specific cleaning composition materials are exemplified in detail hereinafter.

As used herein, “effective amount of enzyme” refers to the quantity of enzyme necessary to achieve the enzymatic activity required in the specific application. Such effective amounts are readily ascertained by one of ordinary skill in the art and are based on many factors, such as the particular enzyme variant used, the cleaning application, the specific composition of the cleaning composition, and whether a liquid or dry (e.g., granular) composition is required, and the like.

As used herein, the phrase “detergent stability” refers to the stability of a detergent composition. In some embodiments, the stability is assessed during the use of the detergent, while in other embodiments, the term refers to the stability of a detergent composition during storage.

The term “improved stability” is used to indicate better stability of enzymes in substrate containing compositions. In preferred embodiments, the enzymes exhibit improved stability in laundry or dishcare detergents with inhibitors during storage, relative to the corresponding formulations without enzyme inhibitors. In preferred embodiments, the enzyme/substrate system exhibit improved stability during storage in laundry or dishcare detergents with inhibitors, relative to the corresponding formulations without enzyme inhibitors.

As used herein, “oxidative stability” refers to the ability of a protein to function under oxidative conditions. In particular, the term refers to the ability of a protein to function in the presence of various concentrations of H₂O₂, peracids and other oxidants. Stability under various oxidative conditions can be measured either by standard procedures known to those in the art and/or by the methods described herein. A substantial change in oxidative stability is evidenced by at least about a 5% or greater increase or decrease (in most embodiments, it is preferably an increase) in the half-life of the enzymatic activity, as compared to the enzymatic activity present in the absence of oxidative compounds.

As used herein, “pH stability” refers to the ability of a protein to function at a particular pH. In general, most enzymes have a finite pH range at which they will function. In addition to enzymes that function in mid-range pHs (i.e., around pH 7), there are enzymes that are capable of working under conditions with very high or very low pHs. Stability at various pHs can be measured either by standard procedures known to those in the art and/or by the methods described herein. A substantial change in pH stability is evidenced by at least about 5% or greater increase or decrease (in most embodiments, it is preferably an increase) in the half-life of the enzymatic activity, as compared to the enzymatic activity at the enzyme's optimum pH. However, it is not intended that the present invention be limited to any pH stability level nor pH range.

As used herein, “thermal stability” refers to the ability of a protein to function at a particular temperature. In general, most enzymes have a finite range of temperatures at which they will function. In addition to enzymes that work in mid-range temperatures (e.g., room temperature), there are enzymes that are capable of working in very high or very low temperatures. Thermal stability can be measured either by known procedures or by the methods described herein. A substantial change in thermal stability is evidenced by at least about 5% or greater increase or decrease (in most embodiments, it is preferably an increase) in the half-life of the catalytic activity of a mutant when exposed to given temperature. However, it is not intended that the present invention be limited to any temperature stability level nor temperature range.

As used herein, the term “chemical stability” refers to the stability of a protein (e.g., an enzyme) towards chemicals that may adversely affect its activity. In some embodiments, such chemicals include, but are not limited to hydrogen peroxide, peracids, anionic detergents, cationic detergents, non-ionic detergents, chelants, etc. However, it is not intended that the present invention be limited to any particular chemical stability level nor range of chemical stability.

As used herein, the terms “purified” and “isolated” refer to the removal of contaminants from a sample. For example, an enzyme of interest is purified by removal of contaminating proteins and other compounds within a solution or preparation that are not the enzyme of interest. In some embodiments, recombinant enzymes of interest are expressed in bacterial or fungal host cells and these recombinant enzymes of interest are purified by the removal of other host cell constituents; the percent of recombinant enzyme of interest polypeptides is thereby increased in the sample.

As used herein, “protein of interest,” refers to a protein (e.g., an enzyme or “enzyme of interest”) which is being analyzed, identified and/or modified. Naturally-occurring, as well as recombinant (e.g., mutant) proteins find use in the present invention.

As used herein, “protein” refers to any composition comprised of amino acids and recognized as a protein by those of skill in the art. The terms “protein,” “peptide” and polypeptide are used interchangeably herein. Wherein a peptide is a portion of a protein, those skilled in the art understand the use of the term in context.

As used herein, “cleaning compositions” and “cleaning formulations” refer to compositions that find use in the removal of undesired compounds from items to be cleaned, such as fabric, dishes, contact lenses, other solid substrates, hair (shampoos), skin (soaps and creams), teeth (mouthwashes, toothpastes) etc. The term encompasses any materials/compounds selected for the particular type of cleaning composition desired and the form of the product (e.g., liquid, gel, granule, or spray composition), as long as the composition is compatible with the oxidase and other enzyme(s) used in the composition, and any reversible enzyme inhibitors in the composition. The specific selection of cleaning composition materials are readily made by considering the surface, item or fabric to be cleaned, and the desired form of the composition for the cleaning conditions during use.

The terms further refer to any composition that is suited for cleaning, bleaching, disinfecting, and/or sterilizing any object and/or surface. It is intended that the terms include, but are not limited to detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish detergents).

Indeed, the term “cleaning composition” as used herein, includes unless otherwise indicated, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the terms “detergent composition” and “detergent formulation” are used in reference to mixtures which are intended for use in a wash medium for the cleaning of soiled objects. In some preferred embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. Indeed, it is intended that in addition to perhydrolase, the term encompasses detergents that contain surfactants, transferase(s), hydrolytic enzymes, oxido reductases, builders, bleaching agents, bleach activators, bluing agents and fluorescent dyes, caking inhibitors, masking agents, enzyme activators, enzyme inhibitors, antioxidants, and solubilizers. In some preferred embodiments, the detergent formulations include, but are not limited to those set forth in U.S. patent application Ser. Nos. 10/576,331 and 10/581,014, as well as WO 05/52161 and WO 05/056782 find use in the present invention. However, it is not intended that the present invention be limited to any particular detergent formulation(s), as any suitable detergent formulation finds use in the present invention.

As used herein, “dishwashing composition” refers to all forms of compositions for cleaning dishware, including cutlery, including but not limited to granular and liquid forms. It is not intended that the present invention be limited to any particular type or dishware composition. Indeed, the present invention finds use in cleaning dishware (e.g., dishes, including, but not limited to plates, cups, glasses, bowls, etc.) and cutlery (e.g., utensils, including but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, “wash performance” of an enzyme refers to the contribution of an enzyme to washing that provides additional cleaning performance to the detergent without the addition of the enzyme to the composition. Wash performance is compared under relevant washing conditions.

The term “relevant washing conditions” is used herein to indicate the conditions, particularly washing temperature, time, washing mechanics, sud concentration, type of detergent and water hardness, actually used in households in a detergent market segment.

The term “improved wash performance” is used to indicate that a better end result is obtained in stain removal from items washed (e.g., fabrics or dishware and/or cutlery) under relevant washing conditions, or that less enzyme, on weight basis, is needed to obtain the same end result relative to another enzyme.

The term “retained wash performance” is used to indicate that the wash performance of an enzyme, on weight basis, is at least 80% relative to another enzyme under relevant washing conditions.

Wash performance of enzymes is conveniently measured by their ability to remove certain representative stains under appropriate test conditions. In these test systems, other relevant factors, such as detergent composition, sud concentration, water hardness, washing mechanics, time, pH, and/or temperature, can be controlled in such a way that conditions typical for household application in a certain market segment are imitated.

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

Cleaning and Detergent Formulations

The detergent compositions of the present invention are provided in any suitable form, including for example, but are not limited to liquids, granules, emulsions, gels, and pastes. When a solid detergent composition is employed, the detergent is preferably formulated as granules. Preferably, the granules are formulated to additionally contain a protecting agent (See e.g., U.S. patent application Ser. No. 07/642,669 filed Jan. 17, 1991, incorporated herein by reference). Likewise, in some embodiments, the granules are formulated so as to contain materials to reduce the rate of dissolution of the granule into the wash medium (See e.g., U.S. Pat. No. 5,254,283, incorporated herein by reference in its entirety). In addition, the enzymes of the present invention find use in formulations in which substrate and enzyme are present in the same granule. Thus, in some embodiments, the efficacy of the enzyme present in the formulation is increased by the provision of high local concentrations of enzyme and substrate (See e.g., U.S. Pat. Appln. Publ. US2003/0191033, herein incorporated by reference). Any suitable formulation and/or formulation system finds use in the present invention (See e.g., U.S. Pat. No. 5,204,015; incorporated herein by reference). Those in the art are familiar with the different formulations which find use as cleaning compositions.

Furthermore, proteins, particularly the stabilized oxidases of the present invention can be formulated into known powdered and liquid detergents having pH between 3 and 12.0, at levels of about 0.001 to about 5% (preferably 0.1% to 0.5%) by weight.

It is contemplated that the stabilized oxidases of the present invention will find use in any suitable cleaning composition, including but not limited to bar and liquid soap applications, dishcare formulations, surface cleaning applications, contact lens cleaning solutions or products, waste treatment, textile applications, pulp-bleaching, disinfectants, skin care, oral care, hair care, etc.

While not essential for the purposes of the present invention, the non-limiting list of adjuncts illustrated hereinafter are suitable for use in the instant cleaning compositions and find use in certain embodiments of the invention, for example to assist or enhance cleaning performance, for treatment of the substrate to be cleaned, or to modify the aesthetics of the cleaning composition as is the case with perfumes, colorants, dyes or the like. It is understood that such adjuncts are provided in addition to the stabilized enzymes of the present invention. The precise nature of these additional components, and levels of incorporation thereof, depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments (See e.g., U.S. Pat. Nos. 5,576,282, 6,306,812, and 6,326,348, herein incorporated by reference). The aforementioned adjunct ingredients may constitute the balance of the cleaning compositions of the present invention.

In some preferred embodiments, the detergent compositions of the present invention employ a surface active agent (i.e., surfactant) including anionic, non-ionic and ampholytic surfactants well known for their use in detergent compositions. Some surfactants suitable for use in the present invention are described in British Patent Application No. 2 094 826 A, incorporated herein by reference. In some embodiments, mixtures surfactants are used in the present invention. For example, a number of known compounds are suitable surfactants useful in compositions comprising the protein mutants of the invention. These include nonionic, anionic, cationic, anionic or zwitterionic detergents (See e.g., U.S. Pat. Nos. 4,404,128 and 4,261,868).

Suitable anionic surfactants for use in the detergent composition of the present invention include linear or branched alkylbenzene sulfonates; alkyl or alkenyl ether sulfates having linear or branched alkyl groups or alkenyl groups; alkyl or alkenyl sulfates; olefin sulfonates; alkane sulfonates and the like. Suitable counter ions for anionic surfactants include alkali metal ions such as sodium and potassium; alkaline earth metal ions such as calcium and magnesium; ammonium ion; and alkanolamines having 1 to 3 alkanol groups of carbon number 2 or 3.

Ampholytic surfactants that find use in the present invention include quaternary ammonium salt sulfonates, betaine-type ampholytic surfactants, and the like. Such ampholytic surfactants have both the positive and negative charged groups in the same molecule.

Nonionic surfactants that find use in the present invention generally comprise polyoxyalkylene ethers, as well as higher fatty acid alkanolamides or alkylene oxide adduct thereof, fatty acid glycerine monoesters, and the like.

In some preferred embodiments, the surfactant or surfactant mixture included in the detergent compositions of the present invention is provided in an amount from about 1 weight percent to about 95 weight percent of the total detergent composition and preferably from about 5 weight percent to about 45 weight percent of the total detergent composition. In various embodiments, numerous other components are included in the compositions of the present invention. It is not intended that the present invention be limited to the specific examples set forth herein. Indeed, it is contemplated that additional compounds will find use in the present invention.

In some embodiments, the cleaning compositions provided herein contain at least one chelating agent. Suitable chelating agents include, but are not limited to copper, iron and/or manganese chelating agents and mixtures thereof. When a chelating agent is used, the cleaning composition typically comprises from about 0.1% to about 15% or even from about 3.0% to about 10% chelating agent by weight of the subject cleaning composition.

In some embodiments, the cleaning compositions of the present invention comprise a deposition aid. Suitable deposition aids include, but are not limited to polyethylene glycol, polypropylene glycol, polycarboxylate, soil release polymers such as polytelephthalic acid, clays such as Kaolinite, montmorillonite, atapulgite, illite, bentonite, halloysite, and mixtures thereof.

In some additional embodiments, the cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject cleaning composition, the dye transfer inhibiting agents are typically present at levels from about 0.0001% to about 10%, from about 0.01% to about 5% or even from about 0.1% to about 3% by weight of the cleaning composition.

In some yet further embodiments, the cleaning compositions of the present invention also contain dispersants. Suitable water-soluble organic materials include the homo- or co-polymeric acids or their salts, in which the polycarboxylic acid comprises at least two carboxyl radicals separated from each other by not more than two carbon atoms.

In some embodiments, these detergent cleaning compositions further include other enzymes that typically provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, pectate lyases, keratinases, reductases, oxidases, oxido reductases, phenoloxidases, lipoxygenases, ligninases, mannanases, pullulanases, tannases, pentosanases, peroxidases, malanases, β-glucanases, arabinosidases, hyaluronidase, chondroitinase, laccase, endoglycosidases, and amylases, or mixtures thereof. A typical combination is cocktail of conventional applicable enzymes like a protease, lipase, cutinase, and/or cellulase in conjunction with amylase.

The addition of proteins to conventional cleaning compositions does not create any special use limitations. In other words, any temperature and pH suitable for the detergent are also suitable for the present compositions, as long as the pH is within the range in which the enzyme(s) is/are active, and the temperature is below the described protein's denaturing temperature. In addition, proteins of the invention find use in cleaning, bleaching, and disinfecting compositions without detergents, again either alone or in combination with a source of hydrogen peroxide, an ester substrate (e.g., either added or inherent in the system utilized, such as with stains that contain esters, pulp that contains esters etc), other enzymes, surfactants, builders, stabilizers, etc. Indeed it is not intended that the present invention be limited to any particular formulation or application.

In some further embodiments, the cleaning compositions of the present invention include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra (methylenephosphonic acid) and water-soluble salts thereof. Such catalysts are disclosed in U.S. Pat. No. 4,430,243, incorporated herein by reference. In some embodiments, the compositions herein utilize a manganese compound for catalysis. Such compounds and levels of use are well known in the art (See e.g., U.S. Pat. No. 5,576,282). In some alternative embodiments, cobalt bleach catalysts useful herein are known (See e.g., U.S. Pat. Nos. 5,597,936, 5,595,967, 5,597,936, and 5,595,967).

In some embodiments, the cleaning compositions further comprise a transition metal complex of a macropolycyclic rigid ligand (“MRL”). As a practical matter, and not by way of limitation, the compositions and cleaning processes herein can be adjusted to provide on the order of at least one part per hundred million of the active MRL species in the aqueous washing medium, and will preferably provide from about 0.005 ppm to about 25 ppm, more preferably from about 0.05 ppm to about 10 ppm, and most preferably from about 0.1 ppm to about 5 ppm, of the MRL in the wash liquor. Preferred transition-metals in the instant transition-metal bleach catalyst include manganese, iron and chromium. Preferred MRL's herein are a special type of ultra-rigid ligand that is cross-bridged such as 5,12-diethyl-1,5,8,12-tetraazabicyclo[6.6.2] hexadecane. Suitable transition metal MRLs are readily prepared by known procedures and known in the art (See e.g., WO 00/332601, and U.S. Pat. No. 6,225,464).

In some embodiments, the cleaning compositions of the present invention comprise one or more detergent builders or builder systems. When a builder is used, the subject cleaning composition typically comprises at least about 1%, from about 3% to about 60% or even from about 5% to about 40% builder by weight of the subject cleaning composition. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

In some embodiments of the present invention, the composition contains from about 0 to about 50 weight percent of one or more builder components selected from the group consisting of alkali metal salts and alkanolamine salts of the following compounds: phosphates, phosphonates, phosphonocarboxylates, salts of amino acids, aminopolyacetates high molecular electrolytes, non-dissociating polymers, salts of dicarboxylic acids, and aluminosilicate salts. Examples of suitable divalent sequestering agents are disclosed in British Patent Application No. 2 094 826 A, the disclosure of which is incorporated herein by reference.

In additional embodiments, compositions of the present invention contain from about 1 to about 50 weight percent, preferably from about 5 to about 30 weight percent, based on the composition of one or more alkali metal salts of the following compounds as the alkalis or inorganic electrolytes: silicates, carbonates and sulfates as well as organic alkalis such as triethanolamine, diethanolamine, monoethanolamine and triisopropanolamine.

In yet additional embodiments of the present invention, the compositions contain from about 0.1 to about 5 weight percent of one or more of the following compounds as antiredeposition agents: polyethylene glycol, polyvinyl alcohol, polyvinylpyrrolidone and carboxymethylcellulose. In some preferred embodiments, a combination of carboxymethyl-cellulose and/or polyethylene glycol are utilized with the composition of the present invention as useful dirt removing compositions.

In some further embodiments of the present invention, bleaching agent(s) such as sodium percarbonate, sodium perborate, sodium sulfate/hydrogen peroxide adduct and sodium chloride/hydrogen peroxide adduct and/or a photo-sensitive bleaching dye such as zinc or aluminum salt of sulfonated phthalocyanine further improves the detergent effects of cleaning/bleaching compositions of the present invention. In additional embodiments, bleach boosters (e.g., TAED and/or NOBS) find use.

In some embodiments of the present invention, bluing agents and/or fluorescent dyes are incorporated in the composition. Examples of suitable bluing agents and fluorescent dyes are disclosed in British Patent Application No. 2 094 826 A, the disclosure of which is incorporated herein by reference.

In some embodiments of the present invention in which the composition is powdered or solid, caking inhibitors are incorporated in the composition. Examples of suitable caking inhibitors include p-toluenesulfonic acid salts, xylenesulfonic acid salts, acetic acid salts, sulfosuccinic acid salts, talc, finely pulverized silica, clay, calcium silicate (e.g., Micro-Cell by Johns Manville Co.), calcium carbonate and magnesium oxide.

In some embodiments, antioxidants, including but not limited to tert-butyl-hydroxytoluene, 4,4′-butylidenebis(6-tert-butyl-3-methylphenol), 2,2′-butylidenebis(6-tert-butyl-4-methylphenol), monostyrenated cresol, distyrenated cresol, monostyrenated phenol, distyrenated phenol and 1,1-bis(4-hydroxy-phenyl)cyclohexane find use in the present invention.

In yet additional embodiments, the compositions of the present invention also include solubilizers, including but not limited to lower alcohols (e.g., ethanol, benzenesulfonate salts, and lower alkylbenzenesulfonate salts such as p-toluenesulfonate salts), glycols such as propylene glycol, acetylbenzene-sulfonate salts, acetamides, pyridinedicarboxylic acid amides, benzoate salts and urea.

In some embodiments, the detergent compositions of the present invention are used in a broad pH range of from acidic to alkaline pH. In a preferred embodiment, the detergent composition of the present invention is used in mildly acidic, neutral or alkaline detergent wash media having a pH of from above 4 to no more than about 12.

In addition to the ingredients described above, perfumes, buffers, preservatives, dyes and the like also find use with the present invention. These components are provided in concentrations and forms known to those in the art.

In some embodiments, the powdered detergent bases of the present invention are prepared by any known preparation methods including a spray-drying method and/or a granulation method. The detergent base obtained particularly by the spray-drying method and/or spray-drying granulation method are preferred. The detergent base obtained by the spray-drying method is not restricted with respect to preparation conditions. The detergent base obtained by the spray-drying method is hollow granules which are obtained by spraying an aqueous slurry of heat-resistant ingredients, such as surface active agents and builders, into a hot space. After the spray-drying, perfumes, enzymes, bleaching agents, inorganic alkaline builders may be added. With a highly dense, granular detergent base obtained such as by the spray-drying-granulation method, various ingredients may also be added after the preparation of the base.

When the detergent base is a liquid, in some embodiments it is a homogeneous solution, while in some alternative embodiments, it is an inhomogeneous dispersion.

In some preferred embodiments, the detergent compositions of the present invention are incubated with fabric (e.g., soiled fabrics), in industrial and household uses at temperatures, reaction times and liquor ratios conventionally employed in these environments. The incubation conditions (i.e., the conditions effective for treating materials with detergent compositions according to the present invention), are readily ascertainable by those of skill in the art.

As indicated above, in some embodiments of the present invention detergents are formulated as a pre-wash in the appropriate solution at an intermediate pH where sufficient activity exists to provide desired improvements softening, depilling, pilling prevention, surface fiber removal and/or cleaning. In some embodiments, at least one surfactant is also used. The remainder of the composition comprises conventional components used in the pre-soak (e.g., diluent, buffers, other enzymes (proteases), etc.) at their conventional concentrations.

In some embodiments, the cleaning compositions of the present invention find use in laundry applications, hard surface cleaning, automatic dishwashing applications, as well as cosmetic applications such as cleaning of dentures, teeth, hair and skin. The enzymes of the present invention also find use in cleaning additive products. The additive product may be, in its simplest form, one or more of the stabilized enzymes of the present invention. Such additive may be packaged in dosage form for addition to a cleaning process. Single dosage forms include but are not limited to pills, tablets, gelcaps, or other single dosage units such as pre-measured powders or liquids. In some embodiments, filler and/or carrier material are included to increase the volume of such composition. Suitable filler or carrier materials include, but are not limited to, various salts of sulfate, carbonate and silicate as well as talc, clay and the like. Filler or carrier materials for liquid compositions may be water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials. In some alternative embodiments, acidic fillers are used to reduce pH.

The cleaning compositions and cleaning additives of the present invention require an effective amount of the stabilized enzymes of the present invention. Typically, the cleaning compositions of the present invention comprise at least 0.0001 weight percent, from about 0.0001 to about 1, from about 0.001 to about 0.5, or even from about 0.01 to about 0.1 weight percent of at least one enzyme of the present invention.

In some embodiments, the cleaning compositions of the present invention comprise a material selected from the group consisting of a peroxygen source, hydrogen peroxide and mixtures thereof, said peroxygen source being selected from the group consisting of:

(i) from about 0.01 to about 50, from about 0.1 to about 20, or even from about 1 to 10 weight percent of a per-salt, an organic peroxyacid, urea hydrogen peroxide and mixtures thereof;

(ii) from about 0.01 to about 50, from about 0.1 to about 20, or even from about 1 to 10 weight percent of a carbohydrate and from about 0.0001 to about 1, from about 0.001 to about 0.5, from about 0.01 to about 0.1 weight percent carbohydrate oxidase; and

(iii) mixtures thereof.

In some embodiments, suitable per-salts include those selected from the group consisting of alkalimetal perborate, alkalimetal percarbonate, alkalimetal perphosphates, alkalimetal persulphates and mixtures thereof.

In some preferred embodiments, the carbohydrate is selected from the group consisting of mono-carbohydrates, di-carbohydrates, tri-carbohydrates, oligo-carbohydrates and mixtures thereof. Suitable carbohydrates include carbohydrates selected from the group consisting of D-arabinose, L-arabinose, D-cellobiose, 2-deoxy-D-galactose, 2-deoxy-D-ribose, D-fructose, L-fucose, D-galactose, D-glucose, D-glycero-D-gulo-heptose, D-lactose, D-lyxose, L-lyxose, D-maltose, D-mannose, melezitose, L-melibiose, palatinose, D-raffinose, L-rhamnose, D-ribose, L-sorbose, stachyose, sucrose, D-trehalose, D-xylose, L-xylose and mixtures thereof.

In some embodiments, suitable carbohydrate oxidases include carbohydrate oxidases selected from the group consisting of aldose oxidase (IUPAC classification EC1.1.3.9), galactose oxidase (IUPAC classification EC1.1.3.9), cellobiose oxidase (IUPAC classification EC1.1.3.25), pyranose oxidase (IUPAC classification EC1.1.3.10), sorbose oxidase (IUPAC classification EC1.1.3.11), hexose oxidase (IUPAC classification EC1.1.3.5), and/or glucose oxidase (IUPAC classification EC1.1.3.4) and mixtures thereof.

In some alternative embodiments, the cleaning compositions of the present invention also include from about 0.01 to about 99.9, from about 0.01 to about 50, from about 0.1 to about 20, or even from about 1 to about 15 weight percent a molecule comprising an ester moiety. Suitable molecules that comprise an ester moiety include, but are not limited to polycarbohydrates that comprise an ester moiety. It is intended that any suitable ester moiety will find use in the present invention.

In some preferred embodiments, the cleaning compositions provided herein are typically be formulated such that, during use in aqueous cleaning operations, the wash water will have a pH of from about 5.0 to about 11.5, or even from about 7.5 to about 10.5. Liquid product formulations are typically formulated to have a pH from about 3.0 and about 9.0. Granular laundry products are typically formulated to have a pH from about 9 to about 11. Techniques for controlling pH at recommended usage levels include the use of buffers, alkalis, acids, etc., and are well known to those skilled in the art.

In some embodiments, when the enzyme(s) of the present invention is/are employed in a granular composition or liquid, it is desirable for the enzyme(s) to be in the form of an encapsulated particle to protect such enzyme from other components of the granular composition during storage. In addition, encapsulation is also a means of controlling the availability of the enzyme(s) during the cleaning process and may enhance performance of the enzyme(s). It is contemplated that any suitable encapsulating material will find use in the present invention. The encapsulating material typically encapsulates at least part of the enzyme(s). Typically, the encapsulating material is water-soluble and/or water-dispersible. Indeed, it is intended that the cleaning compositions of the present invention be formulated into any suitable form and prepared by any process chosen by the formulator (See e.g., U.S. Pat. Nos. 5,879,584, 5,691,297, 5,574,005, 5,569,645, 5,565,422, 5,516,448, 5,489,392, and 5,486,303; all of which are incorporated herein by reference, for non-limiting examples).

In some particularly preferred embodiments, the cleaning compositions provided herein find use in cleaning in situ (e.g., on the surface of a fabric or a hard surface). Typically, at least a portion of the situs is contacted with an embodiment of a cleaning composition provided herein, in neat form or diluted in a wash liquor, and then the situs is optionally washed and/or rinsed. For purposes of the present invention, washing includes but is not limited to, scrubbing, and mechanical agitation. It is contemplated that the fabric comprise any suitable fabric capable of being laundered in normal consumer use conditions. The disclosed cleaning compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 30:1.

Experimental

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); bp (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N′N′-tetraacetylethylenediamine); w/v (weight to volume); v/v (volume to volume); GOX and GOx (glucose oxidase); AOX and AOx (alcohol oxidase); COX and Cox (choline oxidase); HOX and HOx (hexose oxidase); SOX and Sox (sorbitol oxidase); AATCC (American Association of Textile and Coloring Chemists); WFK (wfk Testgewebe GmbH, Bruggen-Bracht, Germany); TestFabric (TestFabric Inc, Pittston Pa.); Warwick Equest (Warwick Equest Ltd., Warwick International, Flintshire, UK); ATCC (American Type Culture Collection, Manassas, Va.); Baker (J. T. Baker, Phillipsburg, N.J.); NAEF (NAEF, Press and Dies, Inc., Bolton Landing, N.Y.); Sigma (Sigma-Aldrich Chemical Co., St. Louis, Mo.); and Minolta (Konica Minolta. Glen Cove, N.Y.).

### EXAMPLE 1

### Stabilization of Glucose Oxidase in AATCC Standard Detergent in the Presence of Glucose and Sodium Hydrogen Sulfite

In this Example, experiments conducted to assess the stabilization of glucose oxidase in the presence of its substrate (i.e., glucose) and an inhibitor (i.e., sodium hydrogen sulfite) is described. In these experiments, AATCC standard detergent (American Association of Textile Chemists and Colorists Heavy Duty Liquid detergent version 2003 without brightener; key components include linear alkane sulfonate, alcohol ethoxylate, propanediol, citric acid, fatty acid, castic soda and water; purchased from TestFabrics) was used.

In these experiments, 100 mM Tris pH 8.3, with 0.005% TWEEN®-100 surfactant used as a positive control. Use of pH 8.3 was based upon the measured pH of AATCC detergent. Three two-ml tubes were weighed with 0.990 g of AATCC detergent (lot # 01282004) and another three tubes with 0.990 g of control buffer. Then, 90 mg (500 mM) of glucose substrate were added to all of the tubes. Next, 100, 50, and 10 mM sodium hydrogen sulfite (MW 106.1) were added to each tube respectively, including the control buffer and AATCC detergent control. All of the tubes were placed on a rotary plate for an hour to allow good mixing and solubilization of glucose into the detergent. Then, 500 PPM (0.5 mg, 14.88 ul) glucose oxidase (OXYGO™ L-5000, 5379 U/ml; 33.6 mg/ml; Genencor) was added to six 2-ml tubes (three with glucose/bisulfite containing control buffers and three with glucose/bisulfite containing AATCC detergent). All of the tubes were then set on the rotary plate (60 rpm) at room temperature. Hydrogen peroxide production was measured using dipsticks (peroxidase/ABTS—Baker Testrips;Baker) at various times—t=0+minutes, 12 minutes, and 30 minutes. The tube containing 100 mM bisulfite with glucose, glucose oxidase in liquid detergent was further monitored for premature generation of hydrogen peroxide further over a period of time starting at 1 hr, 12 hr, 7 days, 12 days and up to 21 days. Results obtained in these experiments are provided in Table 1 (See, Example 2).

At time 0+, the buffer control mixture containing 10 mM bisulfite inhibitor generated 1 PPM H₂O₂, whereas the buffer control mixtures containing 50 or 100 mM bisulfite did not produce any hydrogen peroxide for the various time periods tested (See, Table 1).

At time 0+, the detergent mixtures containing 10 mM or 50 mM bisulfite inhibitor generated >10 PPM H₂O₂, whereas the 100 mM bisulfite-containing buffer control mixture did not produce any hydrogen peroxide for the time periods tested (See, Table 1). These results confirmed that 100 mM bisulfite prevents generation of hydrogen peroxide in a liquid detergent formulation containing 500 mM glucose and 500 PPM glucose oxidase, due to oxidase inhibition. Indeed, no premature hydrogen peroxide generation was observed over 21 days in the liquid detergent formulation containing 100 mM sodium hydrogen sulfite, 500 mM glucose and 500 PPM glucose oxidase.

### EXAMPLE 2

### Generation of Hydrogen Peroxide by Glucose Oxidase in Laundry Wash Solution in the Presence of Sodium Hydrogen Sulfite

In this Example, experiments conducted to assess the generation of hydrogen peroxide by glucose oxidase in the presence of sodium hydrogen sulfite in laundry wash liquor are described. As in Example 1, AATCC standard detergent was used in these experiments.

In these experiments, 100 mM Tris pH 8.3 with 0.005% TWEEN®-100 surfactant was used as a positive control. The choice of pH 8.3 was based upon the measured pH of the AATCC detergent. Three two-ml tubes containing 0.990 g of AATCC detergent (lot # 01282004) and another three tubes with 0.990 g of control buffer were weighed. Then, 90 mg (500 mM) glucose substrate was added to each tube. Then, 100, 50, and 10 mM sodium hydrogen sulfite (MW 106.1) (a reversible inhibitor) were added to each tube respectively (i.e., both control buffer and AATCC detergent-containing tubes). All of the tubes were placed on a rotary plate for an hour to allow good mixing and solubilization of glucose into the detergent. Then, 6 tubes containing five-ml wash water (5 mM HEPES with 6 GPG, pH 8) were prepared. Next, 500 PPM (0.5 mg, 14.88 ul) glucose oxidase (OXYGO™ L-5000, 5379 U/ml; 33.6 mg/ml; Genencor) was added to six 2-ml tubes (three with glucose/bisulfite containing control buffers and three with glucose/bisulfite containing AATCC detergent). All of the tubes were then set on the rotary plate (60 rpm) at room temperature. H₂O₂ production was measured using dipsticks (peroxidase/ABTS), as described in Example 1, for t=0+ minutes, 12 minutes, and 30 minutes. Immediately after addition of the enzyme to the detergent and control mixtures, 10 ul of the mixture were removed and mixed with 5 ml of wash water. All of the tubes were also then checked for hydrogen peroxide using dipsticks at 12 and 30 minutes. With 5 ml wash liquor, the final glucose oxidase enzyme concentration was 1 PPM and the glucose concentration was 1 mM.

The results indicated that the wash liquor control containing buffer generated about 10 PPM hydrogen peroxide for formulation containing 10 mM bisulfite inhibitor, ˜10 PPM for 50 mM bisulfite, and ˜3 PPM H₂O₂ for 100 mM bisulfite at 12 minutes. Thus, these results indicate the reversible character of bisulfite inhibitors (See, Table 1 below, for details).

Wash liquor containing AATCC detergent generated >3 PPM for all three bisulfite inhibitor concentrations at 12 minutes, also confirming the reversible character of the bisulfite inhibitor. In addition, wash liquor with AATCC detergent generated ˜10 PPM for all the three bisulfite inhibitor concentrations at 30 minutes, again confirming the reversible character of the bisulfite inhibitor. These results indicate that sodium bisulfite is a reversible inhibitor suitable for keeping glucose oxidase inhibited in the presence of high substrate concentration. However, upon dilution of the detergent in wash water, the inhibition disappears. It is worth noting that sodium bisulfite is a reversible inhibitor of glucose oxidase in a concentration-dependent manner.

TABLE 1
Determination of Hydrogen Peroxide Generation (mg/l, PPM)

| | Control Buffer | AATCC Detergent |
|---|---|---|
| | w/ Glucose | with Glucose |
| | Oxidase | Oxidase |
| Time (min.) | PPM H₂O₂ | PPM H₂O₂ |
| T = 0+, as is, 500 mM | 1 | >10 |
| glucose 500 PPM oxidase, and | | |
| 10 mM bisulfite | | |
| T = 0+, as is, 500 mM | 0 | 10 |
| glucose, 500 PM oxidase, and | | |
| 50 mM bisulfite | | |
| T = 0+, as is, 500 mM | 0 | 0 |
| glucose, 500 PPM oxidase, and | | |
| 100 mM bisulfite | | |
| T = 12, wash liquor, 1 mM | 3 | 3 |
| glucose, and 1 PPM oxidase | | |
| T = 12, wash liquor, 1 mM | ˜10 | 3 |
| glucose, 1 PPM oxidase, and | | |
| 0.02 mM bisulfite | | |
| T = 12, wash liquor, 1 mM | 3 | 3 |
| glucose, 1 PPM oxidase, and | | |
| 0.1 mM bisulfite | | |
| T = 12, wash liquor, 1 mM | ˜3 | 3 |
| glucose, 1 PPM oxidase, and | | |
| 0.2 mM bisulfite | | |
| T = 30, wash liquor, 1 mM | 10 | 10 |
| glucose, 1 PPM oxidase, and | | |
| 0.02 mM bisulfite | | |
| T = 30, wash liquor, 1 mM | ˜10 | ˜10 |
| glucose, 1 PPM oxidase, and | | |
| 0.1 mM bisulfite | | |
| T = 12, wash liquor, 1 mM | ˜3 | ˜10 |
| glucose, 1 PPM oxidase, and | | |
| 0.2 mM bisulfite | | |
| T = 12, wash liquor, 1 mM | 10 | 10 |
| glucose, and 1 PPM oxidase | | |

### EXAMPLE 3

### Stabilization of Glucose Oxidase in Detergent Containing Glucose and Sodium Metabisulfite

In this Example, experiments conducted to assess the generation of hydrogen peroxide by glucose oxidase in the presence of sodium metabisulfite (a reversible inhibitor of oxidase) in laundry wash liquor are described. As in Examples 1 and 2, AATCC standard detergent was used in these experiments.

In these experiments, 100 mM Tris pH 8.3, with 0.005% TWEEN®-100 surfactant was used as a positive control. As above, the choice of pH 8.3 was based upon the measured pH of the AATCC detergent. Three two-ml tubes were weighed with 0.990 g of AATCC detergent (lot # 01282004) and another three tubes with 0.990 g of control buffer.

Three two-ml tubes containing 0.990 g of AATCC detergent (lot # 01282004) and another three tubes with 0.990 g of control buffer were weighed. Then, 90 mg (500 mM) glucose substrate was added to each tube. Then, 100, 50, and 10 mM sodium metabisulfite were added to each tube respectively (i.e., both control buffer and AATCC detergent-containing tubes). All of the tubes were placed on a rotary plate for an hour to allow good mixing and solubilization of glucose into the detergent. Next, 500 PPM (0.5 mg, 14.88 ul) glucose oxidase (OXYGO™ L-5000, 5379 U/ml; 33.6 mg/ml; Genencor) was added to six 2-ml tubes (three with glucose/metabisulfite containing control buffers and three with glucose/metabisulfite containing AATCC detergent). All of the tubes were then set on the rotary plate (60 rpm) at room temperature. H₂O₂ production was measured using dipsticks (peroxidase/ABTS), as described in Examples 1 and 2, for t=0+ minutes, 12 minutes, and 30 minutes. The tube containing 100 mM bisulfite with glucose, glucose oxidase in liquid detergent was further monitored for premature generation of hydrogen peroxide further over a period of time starting at 1 hr, 12 hr, 7 days, 12 days and up to 21 days. The results are provided in Table 2, below.

At time 0+, the buffer control mixture containing 10 mM metabisulfite inhibitor generated 1 PPM H₂O₂, whereas the buffer control mixtures containing 50 or 100 mM bisulfite did not produce any hydrogen peroxide for the various time periods tested (See, Table 2).

At time 0+, the detergent mixtures containing 10 mM or 50 mM metabisulfite inhibitor generated >10 PPM H₂O₂, whereas the 100 mM bisulfite-containing buffer control mixture did not produce any hydrogen peroxide for the time periods tested (See, Table 2). These results confirmed that 100 mM metabisulfite prevents generation of hydrogen peroxide in a liquid detergent formulation containing 500 mM glucose and 500 PPM glucose oxidase, due to oxidase inhibition. Indeed, no premature hydrogen peroxide generation was observed over 21 days in the liquid detergent formulation containing 100 mM sodium metasulfite, 500 mM glucose, and 500 PPM glucose oxidase.

### EXAMPLE 4

### Generation of Hydrogen Peroxide by Glucose Oxidase in Laundry Wash Solution in the Presence of Sodium Metabisulfite

In this Example, experiments conducted to assess the generation of hydrogen peroxide by glucose oxidase in the presence of sodium metabisulfite (a reversible inhibitor of oxidase) in laundry wash liquor are described. As in the above Examples, AATCC standard detergent was used in these experiments.

In these experiments, 100 mM Tris pH 8.3 with 0.005% TWEEN®-100 surfactant was used as a positive control. The choice of pH 8.3 was based upon the measured pH of the AATCC detergent. Three two-ml tubes containing 0.990 g of AATCC detergent (lot # 01282004) and another three tubes with 0.990 g of control buffer were weighed. Then, 90 mg (500 mM) glucose substrate was added to each tube. Then, 100, 50, and 10 mM sodium metabisulfite were added to each tube respectively (i.e., both control buffer and AATCC detergent-containing tubes). All of the tubes were placed on a rotary plate for an hour to allow good mixing and solubilization of glucose into the detergent. Then, 6 tubes containing five-ml wash water (5 mM HEPES with 6 GPG, pH 8) were prepared. Next, 500 PPM (0.5 mg, 14.88 ul) glucose oxidase (OXYGO™ L-5000, 5379 U/ml; 33.6 mg/ml; Genencor) was added to six 2-ml tubes (three with glucose/bisulfite containing control buffers and three with glucose/bisulfite containing AATCC detergent). All of the tubes were then set on the rotary plate (60 rpm) at room temperature. H₂O₂ production was measured using dipsticks (peroxidase/ABTS), as described in the above Examples, for t=0+ minutes, 12 minutes, and 30 minutes. Immediately after addition of the enzyme to the detergent and control mixtures, 10 ul of the mixture were removed and mixed with 5 ml of wash water. All of the tubes were also then checked for hydrogen peroxide using dipsticks at 12 and 30 minutes. With 5 ml wash liquor, the final glucose oxidase enzyme concentration was 1 PPM and the glucose concentration was 1 mM.

The results indicated that the wash liquor control containing buffer generated about 10 PPM in the presence of 10 mM metabisulfite inhibitor, ˜10 PPM for 50 mM metabisulfite, and ˜3 PPM H₂O₂ for 100 mM metabisulfite at 12 minutes. Thus, these results indicate the reversible character of the metabisulfite inhibitor. (See, Table below for details).

Wash liquor containing AATCC detergent generated >3 PPM for all three metabisulfite inhibitor concentrations at 12 minutes, also confirming the reversible character of the metabisulfite inhibitor. In addition, wash liquor with AATCC detergent generated ˜10 PPM for all the three metabisulfite inhibitor concentrations at 30 minutes, again confirming reversible character of the metabisulfite inhibitor. After 3 weeks of storage in the presence of the inhibitor, upon dilution in wash liquor, the liquid detergent formulations produced 3 PPM hydrogen peroxide in 12 minutes. By 30 minutes ˜10 PPM hydrogen peroxide were produced.

These results indicate that sodium metabisulfite is a reversible inhibitor suitable for keeping glucose oxidase inhibited in the presence of high substrate concentration. However, upon dilution of the detergent in wash water, the inhibition disappears. It is worth noting that sodium metabisulfite is a reversible inhibitor of glucose oxidase in a concentration-dependent manner.

In addition to the sodium metabisulfite and sodium bisulfite described in these Examples, other inhibitors were tested for glucose oxidase inhibition. The same methods as described in these Examples were used. The results indicated that 1 M sodium fluoride or thiosulfate produced a small degree of glucose oxidase inhibition. However, it was determined that 2 M hydroxylamine can stabilize premature hydrogen peroxide generation in detergent containing 1 M glucose and 500 PPM glucose oxidase.

TABLE 2
Determination of H₂O₂

| | Control Buffer | AATCC Detergent |
|---|---|---|
| | (glucose oxidase) | (glucose oxidase) |
| Time (Min.) | H₂O₂ PPM | H₂O₂ PPM |
| T = 0+, 12, 30, as is, 500 mM | 0 | 0 |
| glucose and 500 PPM enzyme, | | |
| 100 mM sodium metabisulfite | | |
| T = 12, wash liquor, 1 mM | 3 | 3 |
| glucose, 1 PPM glucose | | |
| oxidase, no inhibitor | | |
| T = 12, wash liquor, 1 mM | 1 | 3 |
| glucose and 1 PPM enzyme, | | |
| 0.2 mM sodium metabisulftie | | |
| T = 30, wash liquor, 1 mM | 3 | ˜10 |
| glucose and 1 PPM enzyme, | | |
| 0.2 mM sodium metabisulfite | | |
| T = 30, wash liquor, 1 mM | 10 | 10 |
| glucose oxidase, no inhibitor | | |
| T = 60, wash liquor, 1 mM | 10 | >10 |
| glucose, 1 PPM glucose | | |
| oxidase, 0.2 mM sodium | | |
| metabisulfite | | |

### EXAMPLE 5

### Stabilization of Alcohol Oxidase in Detergent Containing Substrate and an Inhibitor

In this Example, experiments conducted to assess the stabilization of alcohol oxidase in the presence of its substrate (ethanol) and an inhibitor (e.g., sodium metabisulfite, bisulfite or thiosulfate) is described. As in the above Examples, AATCC standard detergent was used in these experiments.

In these experiments, the stability of alcohol oxidase obtained from Hansunela sp., (100 U/ml. 22 U/mg, 13 mg total solid in vial, 7.7 U/mg solid; Sigma) in AATCC liquid detergent was tested. In these experiments, 10 U of the alcohol oxidase were used in liquid detergent stock for each experiment. For testing, 1M ethanol was mixed into the detergent (46 mg ethanol in 990 mg detergent). The presence of 1M Ethanol did not affect the overall appearance of the liquid detergent. Sodium hydrogen sulfite (NaHSO₃), sodium metabisulfite (Na₂S₂O₅), and sodium thiosulfate (Na₂S₂O₃) were tested as reversible inhibitors of the alcohol oxidase. The experiments were conducted as described above in Examples 1 and 3.

Alcohol oxidase enzyme was found to be stable in liquid AATCC detergent for the period of time (120 minutes) tested in presence of the ethanol substrate and inhibitors. Thiosulfate was found to be a week inhibitor of the alcohol oxidase, while sodium hydrogensulfite and metabisulfite were found to be reversible inhibitors of alcohol oxidase at the 100 mM concentration tested. At 100 mM concentrations, sodium hydrogen sulfite and metabisulfite were able to stop premature generation of H₂O₂ in AATCC detergent stock for the period of investigation (120 minutes), as indicated in Tables 3 and 4.

### EXAMPLE 6

### Generation of H₂O₂ by Alcohol Oxidase in Laundry Wash Solutions Containing Ethanol and a Reversible Inhibitor

In this Example, experiments conducted to assess the generation of hydrogen peroxide by alcohol oxidase in the presence of sodium metabisulfite, bisulfite and thiosulfate (reversible inhibitors of alcohol oxidase) in laundry wash liquor are described. As in the above Examples, AATCC standard detergent was used in these experiments.

The stability and activity of the alcohol oxidase enzyme (described in Example 5) in AATCC liquid detergent (Sigma, 100 U/ml. 22 U/mg, 13 mg total solid in vial, 7.7 U/mg solid) was tested upon dilution into wash liquor. In these experiments, 10 U of alcohol oxidase were used in liquid detergent stock for each experiment. In addition, 1M ethanol (substrate) was mixed in detergent (46 mg ethanol in 990 mg detergent). Upon 500× dilution, the wash liquor contained 2 mM ethanol, to generates a maximum of 2 mM H₂O₂ and a final alcohol oxidase dosage in the wash liquor of 0.02 U. Sodium hydrogen sulfite (NaHSO₃), sodium metabisulfite (Na₂S₂O₅), and sodium thiosulfate (Na₂S2O₃) were tested as reversible inhibitors. In these experiments, 10 ul of final detergent mix or control mix were added to 5 ml of wash liquor. The same methods as described in Examples 2 and 4 were used in these experiments.

Upon dilution in wash liquor, hydrogen peroxide was formed in the presence of detergent containing alcohol oxidase, ethanol and an inhibitor (e.g., sodium hydrogen sulfite or sodium metabisulfite), as indicated in Tables 3 and 4.

TABLE 3
Determination of Hydrogen Peroxide
Concentration in Liquid Detergent

| | Control Buffer | AATCC Detergent |
|---|---|---|
| | Alcohol Oxidase | Alcohol Oxidase |
| Time (min.) | H₂O₂ PPM | H₂O₂ PPM |
| T = 0+, as is, 1 M ethanol and | 10 | 10 |
| 10U enzyme, no inhibitor | | |
| T = 0+, as is 1 M ethanol, 10U | 0 | 0 |
| enzyme, 100 mM sodium | | |
| metabisulfite (detergent | | |
| thickens) | | |
| T = 0+, as is, 1 M ethanol, 10U | ˜10 | 3 |
| enzyme, 100 mM sodium | | |
| thiosulfate | | |
| T = 0+, as is, 1 M ethanol, 10U | 0 | 0 |
| enzyme, 100 mM sodium | | |
| hydrogen sulfite | | |
| T = 12, as is, 1 M ethanol and | 30 | 30 |
| 10U enzyme, no inhibitor | | |
| T = 12, as is 1 M ethanol, 10U | 0 | 0 |
| enzyme, 100 mM sodium | | |
| metabisulfite | | |
| T = 12, as is, 1 M ethanol, 10U | 0 | 0 |
| enzyme, 100 mM sodium | | |
| hydrogen sulfite | | |
| T = 30, as is, 1 M ethanol and | 30 | 30 |
| 10U enzyme, no inhibitor | | |
| T = 30, as is 1 M ethanol, 10U | 0 | 0 |
| enzyme, 100 mM sodium | | |
| metabisulfite | | |
| T = 30, as is, 1 M ethanol, 10U | 0 | 0 |
| enzyme, 100 mM sodium | | |
| hydrogen sulfite | | |
| T = 120, as is, 1 M ethanol and | 30 | 30 |
| 10U enzyme, no inhibitor | | |
| T = 120, as is 1 M ethanol, 10U | 0 | 0, |
| enzyme, 100 mM sodium | | (>10--thiosulfate) |
| hydrogen sulfite or | | |
| metabisulfite or thiosulfate | | |

TABLE 4
Determination of Hydrogen Peroxide Concentration in Wash Liquor

| | Control Buffer | AATCC Detergent |
|---|---|---|
| | Alcohol Oxidase | Alcohol Oxidase |
| Time (min.) | H₂O₂ PPM | H₂O₂ PPM |
| T = 0+, wash liquor, 2 mM | 0 | |
| ethanol and 0.02U enzyme, no inhibitor | | |
| T = 0+, wash liquor, 2 mM | 0 | |
| ethanol, 0.02U enzyme, 0.2 mM | | |
| sodium metabisulfite | | |
| T = 0+, wash liquor, 2 mM | 0 | 0 |
| ethanol, 0.02U enzyme, 0.2 mM | | |
| sodium thiosulfate | | |
| T = 0+, wash liquor, 2 mM | 0 | 0 |
| ethanol, 0.02U enzyme, 0.2 mM | | |
| sodium hydrogen sulfite | | |
| T = 12, wash liquor, 2 mM | 1 | 0 |
| ethanol and 0.02U enzyme, no inhibitor | | |
| T = 12, wash liquor, 2 mM | 0 | 0 |
| ethanol, 0.02U enzyme, 0.2 mM | | |
| sodium metabisulfite | | |
| T = 12, wash liquor, 2 mM | 0 | 0 |
| ethanol, 0.02U enzyme, 0.2 mM | | |
| sodium hydrogen sulfite | | |
| T = 30, wash liquor, 2 mM | 0 | 0 |
| ethanol and 0.02U enzyme, no inhibitor | | |
| T = 30, wash liquor, 2 mM | 0 | 0 |
| ethanol, 0.02U enzyme, 0.2 mM | | |
| sodium metabisulfite or hydrogen | | |
| sulfite | | |
| T = 30, wash liquor, 2 mM | 0 | 0 |
| ethanol, 0.02U enzyme, 0.2 mM | | |
| sodium thiosulfate | | |
| T = 120, wash liquor, 2 mM | ˜10 | ˜10 |
| ethanol and 0.02U enzyme, no inhibitor | | |
| T = 120, wash liquor, 2 mM | 1 (hydrogen sulfite) | >3 (hydrogen sulfite) |
| ethanol, 0.02U enzyme, 0.2 mM | 0 (metabisulfite) | 1 (metabisulfite) |
| sodium metabisulfite or hydrogen | | >3 (thiosulfate) |
| sulfite or thiosulfate | | |

In additional experiments, the ability of 10 mM CuSO₄ to stabilize premature H₂O₂ generation in detergent containing 1 M Ethanol and 10 U of alcohol oxidase (Candida sp.; Sigma) was also assessed.

### EXAMPLE 7

### Stabilization of Choline Oxidase in Detergent Containing Choline and an Inhibitor

In this Example, experiments conducted to assess the stabilization of choline oxidase in the presence of its substrate (i.e., choline) and an inhibitor (i.e., sodium bisulfite and 2-amino,2-methyl,1-propanol) is described. In these experiments, AATCC standard detergent was used.

The experiments were conducted as described in Example 1. Choline oxidase produces two moles of H₂O₂ per mole of choline. The results obtained in these experiments confirmed that choline oxidase was stable in AATCC detergent over 24 hour period tested in presence of choline and inhibitors. The inhibitor 2-amino,2-methyl,1-propanol (AMP) is a reversible inhibitor for choline oxidase and stops premature regeneration of H₂O₂ in detergent when used at 200 mM. Sodium hydrogen sulfite was found to be a reversible inhibitor of choline oxidase at the 100 mM concentration tested. Sodium hydrogen sulfite (100 mM concentration) was also able to stop premature generation of H₂O₂ in AATCC detergent stock. Upon dilution in wash liquor, detergent containing choline oxidase, choline chloride and an inhibitor (e.g., sodium hydrogen sulfite or 2-amino,2-methyl,1-propanol) produced H₂O₂ over time, as indicated in Table 5.

TABLE 5
Determination of H₂O₂ in Liquid Detergent and Wash Liquor

| | | AATCC |
|---|---|---|
| | Control Buffer | Detergent |
| | Choline oxidase | Choline oxidase |
| Time (min.) | H₂O₂ PPM | H₂O₂ PPM |
| T = 0+, 1 M choline, 1U | 10 | 10 |
| enzyme, no inhibitor | | |
| T = 0+, 1 M choline, 1U | 0 | 0 |
| enzyme, 100 mM AMP inhibitor | | |
| T = 12, 1 M choline, 1U | 1 | 1 |
| enzyme, 100 mM AMP inhibitor | | |
| T = 12, 1 M choline, 1U or | 0 | 0 |
| 10U enzyme, 200 mM AMP | | |
| (pH ˜9.3) | | |
| T = 0+, 12, 30 and 60, | 1, 3, | 1, 3, |
| wash liquor, 2 mM choline | 3, 10 | 3, 10 |
| chloride, 0.02U enzyme, 0.4 | | |
| mM AMP inhibitor | | |
| T = 0+, 12, 30 and 60, | 0 | 0 |
| 1M choline chloride, 10U | | |
| choline oxidase, 100 mM sodium | | |
| bisulfite inhibitor (pH7) | | |
| T = 0+, 12, 30 and 60, | 1, 10, | 1, 10, |
| wash liquor, 2 mM choline | >10, 30 | >10, 30 |
| chloride, 0.02U choline | | |
| oxidase, 0.2 mM sodium | | |
| bisulfite inhibitor (pH7) | | |

### EXAMPLE 8

### Stability/Performance of Glucose Oxidase and Hexose Oxidase in Detergent Containing Sodium Bisulfite and Glucose

In this Example, experiments conducted to assess the stability of glucose oxidase and hexose oxidase in detergent containing sodium bisulfite and glucose are described. Additional experiments to assess the performance of these enzymes on stained swatches are also described.

In four 250 ml glass bottles, 100 gram of AATCC liquid detergent was mixed with 9 gram of glucose and stirred for 30 minutes, in order to dissolve the glucose in the detergent. Then, 2.12 grams of sodium bisulfite were added to two bottles dissolved in the detergent. Then, 15,000 Units of glucose oxidase were added to two bottles (one with and one without bisulfite) and similarly 15,000 Units of hexose oxidase were added to the other two bottles (one with and one without bisulfite). All of the four liquid detergent formulations were kept at room temperature and were assayed for their efficacy of stain removal using disk swatches in 12 well plates over a period of 7 days.

Blueberry and tea stained swatches (CS15-004, CS3; TestFabric) were cut into 15 mm circles with a textile punch press (Model 93046; NAEF) equipped with a ⅝″ die cutter. Single disks were placed into each well of a 24-well microplate (Costar). One (1) ml of washing solution pH 10.0 containing per liter, 1.5 ml AATCC HDL detergent, 10 mM sodium carbonate, 75 mM glucose, 6 gpg hardness (diluted from stock 15000 gpg hardness solution containing 1.735 M calcium chloride and 0.67 M magnesium chloride), and 0.05% TAED (tetraacetylethylenediamine, Fluka) was added to each well. Five (5) microliters of 5-7 days old formulated glucose oxidase with or without sodium bisulfite were added with a positive displacement pipette to 4 wells in one column. Control wells (8) contained no enzyme.

The microplate was covered with its plastic lid and incubated at 37° C. with 100 rpm gentle rotation. After 5 hr, the supernatants were removed by aspiration and each well was washed twice with 1.5 ml of Dulbecco's PBS pH 7.3, and twice times with 1.5 ml of distilled water. Each disk was removed from its well and dried overnight in air.

Disks were inspected visually and analyzed with a Minolta Reflectometer CR-200 calibrated on a standard white tile. The average L values were calculated. Surface reflectance of a textile is measured as Lambertian reflectance called the “L-value” (the ratio of reflected light to incident light, generally expressed in percentage) at the surface of a material so thick that the reflectance does not change with increasing thickness (i.e., the intrinsic reflectance of the surface), irrespective of other parameters such as the reflectance of the rear surface. The L-value is measured by measuring reflectance using the above mentioned reflectometer, as was the percent soil release (% SR=100%×(Final reflectance−Initial reflectance)/(Reflectance of a white standard−Initial reflectance).

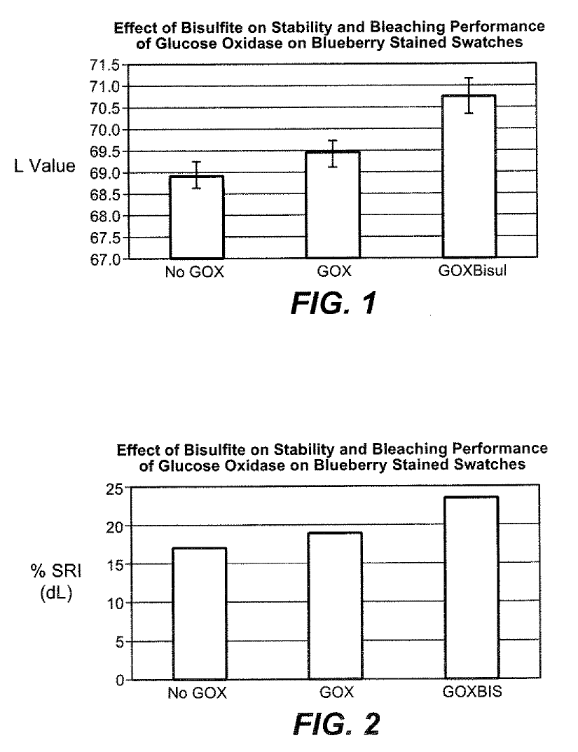

After 5 days, glucose oxidase formulated without bisulfite was yellow, had significant (>30 mg/L) hydrogen peroxide in the formulated sample, showed minimal hydrogen peroxide production activity (1 mg/L) during the disk test and had a bleaching performance that was not statistically different from the no enzyme control. In contrast, after 7 days, glucose oxidase formulated with bisulfite was white, showed robust (>100 mg/L) hydrogen peroxide production during the disk test, and performs significantly better than both the control and the glucose oxidase without bisulfite. The same results were observed after 14 days. The results are provided in Table 6, and 7 and are shown in FIGS. 1 and 2. These results indicate that bisulfite-stabilized glucose oxidase bleaches blueberry-stained disks and tea stained discs significantly better than the control (i.e., no enzyme) or unstabilized glucose oxidase.

TABLE 6
Performance of Oxidase-Containing Liquid Detergent for Blueberry
Stain Removal at Day 5 for Unstabilized Glucose Oxidase and
Day 7 for Bisulfite-stabilized Glucose Oxidase

| | | 5 Days | 7 Days |
|---|---|---|---|
| | No Glucose | Glucose | Bisulfite-stabilized |
| | Oxidase | Oxidase | Glucose Oxidase |
| % Stain Removal (dL) | 17.2 | 18.8 | 23.2 |
| L Value | 68.93 | 69.42 | 70.74 |
| Standard Deviation | 0.33 | 0.32 | 0.42 |

TABLE 7
Performance of Oxidase-Containing Liquid Detergent for Tea
Stain Removal at Day 14 for Unstabilized Glucose Oxidase
and Day 16 for Bisulfite-stabilized Glucose Oxidase

| | | 14 Days | 16 Days |
|---|---|---|---|
| | No Glucose | Glucose | Bisulfite-stabilized |
| | Oxidase | Oxidase | Glucose Oxidase |
| % Stain Removal (dL) | 6.9 | 12 | 32.2 |
| L Value | 75.14 | 76.2 | 80.2 |
| Standard Deviation | 0.37 | 0.36 | 0.35 |

### EXAMPLE 9

### Tergotometer Testing of Two-Month Stability/Performance of Glucose Oxidase in Detergent Containing Sodium Bisulfite and Glucose

In this Example, experiments conducted to assess the stability of glucose oxidase (GOX) in liquid detergent containing sodium bisulfite and glucose is described. Additional experiments to assess the performance of these enzymes on multiple stained swatches are also described.

In two 250 ml glass bottles, 100 grams of AATCC liquid detergent was mixed with 9 grams of glucose and stirred for 30 minutes, in order to dissolve the glucose in the detergent. Then, 2.12 grams of sodium bisulfite (Sigma Aldrich # 243973) was added to one bottle and dissolved in the detergent. Then, 15,000 Units of glucose oxidase (HPL5000, 5379 U/ml; Genencor) were added to both bottles (i.e., the bottle with and the bottle without bisulfite). Both liquid detergent formulations were kept at room temperature for two months and were assayed for their efficacy of stain removal using multistained swatches (Warwick-Equest) in a Tergotometer.

In these tergotometer tests, 950 ml of MilliQ Water were added to pot 1 and 4 and 950 ml of MilliQ water was added to pot 2 & 3. Fifty ml of 1.5 M glucose solution were added to pot 1 & 4. Three ml of fresh AATCC detergent were added to Pot 1 & 4, whereas pot 2 and 3 received 2 month-old formulated ATCC detergent with GoX (i.e., without and with bisulfite) as described above. A final concentration of 2 mM bisulfite was made in pot 1 & 4 by adding a sodium bisulfite stock solution (1M). The water hardness was maintained at 6 gpg (i.e., North American wash conditions). The final TAED concentration in all the pots was kept at 0.05% along with addition of sodium carbonate to bring the pH between 8.5 and 9.15. Twenty-four multistained swatches were pre-read and six swatches were added to each pot and stirred at 125 RPM. Then, 100 ul of stock glucose oxidase were added to pot 4, whereas no glucose oxidase was added to pot 1. Thus, pots 1 and 4, respectively, were used as negative and positive controls. The tergotometer experiment was run for 90 minutes at 30 C. After tergotometer testing, the swatches were washed (3×) with cold tap water, spin dried, and then dried overnight at RT. All of the swatches were steam-pressed and then assessed using a Minolta reflectometer.

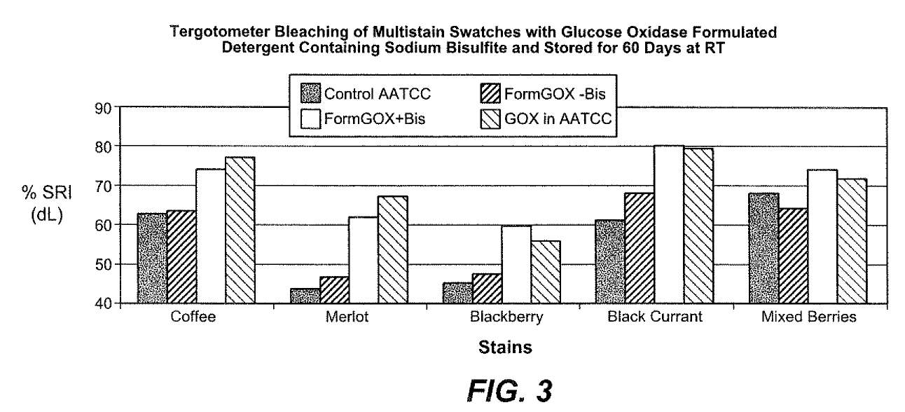

The tergotometer studies confirmed the stability of bisulfite formulated GOX and also confirmed its superiority in bleach performance to GOX formulated without bisulfite and the control on coffee, merlot, blackberry, blackcurrant, and mixed berry stains (See, Table 8 and FIG. 3).

TABLE 8
Bleaching of Multistain Swatches with GoX Formulated With and Without Bisulfite

| | Pot 1 | | Pot 2 | | Pot 3 | | Pot 4 | |
|---|---|---|---|---|---|---|---|---|
| | Control AATCC | | GoX without | | GoX with | | GoX Positive | |
| | Detergent | | bisulfite | | bisulfite | | Control | |
| Stain | % SRI (dL) | Std. Dev. | % SRI (dL) | Std. Dev. | % SRI (dL) | Std. Dev. | % SRI (dL) | Std. Dev. |
| Coffee | 63.08 | 3.16 | 63.52 | 1.73 | 74.88 | 1.89 | 77.85 | 0.78 |
| Merlot | 43.44 | 2.16 | 46.93 | 2.68 | 61.93 | 2.20 | 67.79 | 2.13 |
| Blackberry | 44.69 | 2.61 | 47.24 | 2.81 | 59.75 | 1.71 | 56.04 | 1.73 |
| Black Currant | 60.87 | 2.29 | 68.00 | 2.57 | 80.06 | 1.23 | 79.50 | 1.18 |
| Mixed Berry | 67.61 | 2.12 | 63.56 | 4.76 | 74.29 | 3.10 | 71.33 | 2.66 |

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Having described the preferred embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whetheror not the excised material is specifically recited herein.

We claim:

1. A stabilized oxidase composition comprising said oxidase and a stabilizer.

2. The composition of claim 1, wherein said oxidase is selected from glucose oxidase, sorbitol oxidase, choline oxidase, hexose oxidase, and alcohol oxidase.

3. The composition of claim 1, further comprising at least one substrate for said oxidase.

4. The composition of claim 3, wherein said substrate is selected from glucose, lactate, sorbitol, choline, glycerol, ethylene glycol, propylene glycol, and ethanol.

5. The composition of claim 1, wherein said stabilizer comprises at least one oxidase inhibitor.

6. The composition of claim 5, wherein said stabilizer comprises at least one sulfite.

7. The composition of claim 6, wherein said at least one sulfite is selected from sodium hydrogen sulfite, sodium metabisulfite, and/or sodium bisulfite.

8. The composition of claim 5, wherein said stabilizer is selected from thiosulfate and 2-amino-2methyl-1-propanol.

9. The composition of claim 1, wherein said composition is a cleaning, bleaching or disinfecting composition.

10. The composition of claim 9, wherein said detergent is a laundry detergent or a dish detergent.

11. The composition of claim 10, wherein said detergent is selected from powder, liquid and gel detergents.

12. The composition of claim 1, wherein said composition is a detergent additive or a pretreatment product.

13. The composition of claim 1, further comprising a bleach activator or a bleach precursor.

14. The composition of claim 13, wherein said activator is selected from peracid precursors, metal complexes, peroxidases, and an acyl transferase-substrate system.

15. The composition of claim 1, further comprising at least one enzyme selected from proteases, amylases, pectinases, pectate lyases, lipases, mannanases, cellulases, esterases, cutinases, oxidoreductases, hemicellulases, and carbohydrases.

16. The composition of claim 1, further comprising at least one adjunct ingredient selected from surfactants, builders, whitening agents, antimicrobial agents, polymers, solvents, salts, buffering agents, chelating agents, dye transfer inhibiting agents, deposition aids, dispersants, enzymes, enzyme stabilizers, catalytic materials, bleach activators, bleach boosters, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, pigments and mixtures thereof.

17. A method for producing bleach species in a wash liquor comprising the step of adding said composition of claim 1 to said wash liquor.

18. The method of claim 16, wherein said bleaching species is peroxide or a bleaching system that can be activated by peroxide.
